(19) United States
(12) Patent Application Publication
(10) Pub. No.: US 2016/0304431 A1
(43) Pub. Date: Oct. 20, 2016

(54) A PROCESS FOR PREPARING SUCCINIC ACID AND SUCCINATE ESTER

(71) Applicants: Thidarat TOSUKHOWONG, Billerica, MA (US); MYRIANT CORPORATION, Woburn, MA (US)

(72) Inventor: Thidarat Tosukhowong, Billerica, MA (US)

(73) Assignee: MYRIANT CORPORATION, Woburn, MA (US)

(21) Appl. No.: 15/101,042

(22) PCT Filed: Dec. 5, 2014
(86) PCT No.: PCT/US2014/068840

§ 371 Date: Jun. 2, 2016

Related U.S. Application Data
(60) Provisional application No. 61/912,580, filed on Dec. 6, 2013.

(51) Int. Cl. C07C 51/02; C12P 7/46; C07C 67/08; C07C 67/54; C07C 51/43; C07C 51/47

(52) U.S. Cl. CPC C07C 51/02; C07C 51/43; C07C 51/47; C07C 67/08; C07C 67/54; C12P 7/46

(57) ABSTRACT

This invention relates to a process for preparing succinic acid and succinate ester from a succinic acid salt in fermentation broth. In the first stage of this invention, renewable carbon resources are utilized to produce succinic acid through biological fermentation. The succinic acid salt in the fermentation process is subjected to double displacement reaction with a strong acid leading to release of succinic acid. Succinic acid is recovered by fractional crystallization integrated with simulated moving bed chromatography to produce succinic acid and succinate ester.

### CROSS-REFERENCE TO RELATED APPLICATION

This application claims the priority to the U.S. Provisional Application Ser. No. 61/912,580, filed on Dec. 6, 2013.

### FIELD OF THE INVENTION

The present invention is in the field of producing specialty and commodity organic chemicals using biocatalysts that have been modified to increase their ability to use renewable carbon resources. More specifically, the present invention is related to producing succinic acid and succinate ester from a succinic acid salt obtained from renewable carbon resources through biological fermentation involving biocatalysts.

### BACKGROUND OF THE INVENTION

Dialkyl succinate, 1,4-butanediol (BDO), gamma-butyrolactone (GBL), tetrahydrofuran (THF), and crystalline succinic acid are useful industrial chemicals. For example, di-(ethylhexyl) succinate is a good low-volatile organic solvent for paint application, while dimethyl succinate finds many specialty applications, such as ingredient in cosmetics and fragrance. BDO is currently used as an industrial solvent, in the manufacture of plastics and polyesters, and is a precursor to useful chemicals like GBL and THF. It is a protic polar solvent, which is miscible with water. The current global market for BDO is about 3 billion pounds per year, almost exclusively produced from petrochemical processes. GBL is used as a solvent and it is useful in replacing environmentally harmful chlorinated solvents. GBL is used as an intermediate in the preparation of pyrrolidones used as a raw material in the manufacture of herbicides, rubber additives, and pharmaceuticals. THF is an aprotic, miscible solvent used in organic chemistry. It is also widely used in the production of resins and polymers.

The typical process to produce BDO starts from petrochemical derived acetylene. Acetylene is reacted with formaldehyde using Reppe chemistry. The resulting 1,4-butynediol is then hydrogenated to form BDO. There are several other chemical routes to synthesize BDO, but one of the most economical routes uses butane as a raw material. In the first step of this process, butane is oxidized to produce maleic anhydride. Then maleic anhydride can be converted to BDO via the BP/Lurgi Geminox process or the Davy Technology Process. The former process recovers maleic anhydride as maleic acid and performs liquid-phase hydrogenation to produce a mixture of BDO with THF and/or GBL. In the Davy Technology Process, maleic anhydride is esterified to dimethyl maleate, which is then vaporized and fed to a vapor-phase hydrogenation system to produce dimethyl succinate. Dimethyl succinate undergoes hydrogenolysis reaction to produce GBL and BDO, which can be further converted into THF. These products are separated by distillation and methanol is recycled back to the esterification reactor.

The conventional process of producing BDO, GBL, and THF is not a sustainable process, since the raw material is derived from petroleum. One of the possible pathways to derive a bio-based BDO is by esterifying the bio-succinic acid to dialkyl succinate, followed by a hydrogenation step to produce BDO, GBL, and THF.

Recently, there has been a significant advancement in the production of succinic acid via fermentation of renewable sugar using biocatalyst, such as E. coli, Actinobacillus succinogens and Mannheimia succiniproducens. Bio-succinic acid can be polymerized with BDO to form a biodegradable polybutylene succinate (PBS) polymer. Furthermore, bio-succinic acid derived from fermentation broth can be esterified with alcohol to make dialkylsuccinate and then subjected to vapor-phase hydrogenation to yield BDO, GBL, and THF. The route from bio-succinic acid to BDO via an alkyl ester of succinic acid has a significant advantage in reducing the carbon dioxide (CO₂) footprint over the conventional petro-based BDO. This is because of the fact that the microorganism, such as E. coli, directly consumes CO₂ in order to make succinic acid as shown in Eq. [1]. An E. coli strain producing succinic acid through a fermentation process requires about 0.5 mole of CO₂ to make each mole of succinic acid. As a result, there is a strong need for a low-cost and flexible process to producing ester of succinic acid from fermentation broth.

7C₆H₁₂O₆ (dextrose)+6CO₂→12C₄H₆O₄ (succinic acid)+6H₂O Eq. [1]

The succinic acid fermentation process using bacteria is typically maintained at near neutral pH by adding base to the fermentor as succinic acid is being produced. As a result, the product at the end of fermentation is in the form of a succinate salt. To convert succinate salt back to succinic acid, several methods have been proposed. Similarly, several methods have been proposed for the esterification of the succinic acid recovered from fermentation broth as well as the conversion of succinic acid ester into BDO, GBL, and THF through hydrogenation reactions. The present invention provides an integrated process for recovering succinic acid with minimal impurities from fermentation broth and alkylating it to produce succinate ester.

Numerous investigations and efforts on designing an adequate downstream process for the recovery of succinic acid and its salt have been conducted and reviewed. The core separation technologies of different downstream routes include lime precipitation, chromatography (simulated moving bed), electro dialysis (ED), reactive extraction, adsorption (ion exchange resin or zeolite), crystallization, etc. Most of these approaches are principally functional in lab and even at pilot scale, but industrial success will depend on scalability, operability, robustness, yield and costs. The objective of the present invention is to provide a novel process for recovering succinic acid and succinate ester from fermentation broth containing ammonium succinate which is cost-effective when scaled to industrial level.

U.S. Pat. No. 5,168,055 disclosed a process for recovering succinic acid from the fermentation broth containing calcium succinate. As per this process, sulfuric acid is added to the fermentation broth containing calcium succinate to yield succinic acid and calcium sulfate (gypsum). Calcium sulfate resulting from this process for succinic acid recovery has very little to no commercial value and typically ends up as a landfill waste stream from the process.

U.S. Pat. Nos. 5,034,105 and 5,143,834 disclosed an electro dialysis (ED) process to split succinic acid salt in the fermentation broth to yield a base and succinic acid solution. Such a process has an advantage that no sulfate by-products are generated. However, electro dialysis membrane is often subject to fouling from various proteins, macromolecules, and multivalent ions in the broth, leading to a very high replacement cost.

WO2011/160760 and U.S. Patent Application Publication No. 2013/0096343 disclosed a method for recovering succinic acid from a fermentation broth comprising ammonium succinate. This method comprises an acidification step to produce a mixture of succinic acid and ammonium sulfate, followed by a simulated moving bed (SMB) chromatography step to separate succinic acid from ammonium sulfate. The succinic acid stream from simulated moving bed chromatography is further purified by nanofiltration, and/or activated carbon adsorption, and/or ion-exchange steps. The solution is then evaporated and crystallized to produce pure succinic acid.

The reactive extraction method for recovering carboxylic acid from fermentation broth containing either carboxylic acid or carboxylic acid salt has been described in a number of U.S. patents. One issue associated with reactive extraction process is the challenge faced in recovering solvents at industry scale.

More than 25 sorbents were tested for uptake of succinic acid from aqueous solutions by Davison et al (2004). The best resins have a capacity of about 0.06 g of succinic acid/g of resin at moderate concentrations (1-5 g/L) of succinic acid. 70% recovery was achieved using hot water regeneration, and this regeneration was not stable over 10 cycles in the column. Alternative regeneration schemes using acid and base will increase chemical consumptions and generate more waste. Resin based separation is also subjected to fouling if fermentation broth is not intensively pretreated.

Crystallization is one of the oldest separation and purification techniques known to mankind. Crystallization could be regarded as not only the final purification step but also the first recovery step for the downstream separation of succinic acid. Due to its robustness, operability, and cost effectiveness, crystallization and precipitation from solutions are responsible for 70% of all solid materials produced by the chemical industry.

WO2011/123269 discloses a process for making THF, GBL, and BDO from salt of diammonium succinate broth. The process involves boiling broth at above atmospheric pressure and at temperature of between 100-300° C. The objective of this process was to form overhead product containing water and ammonia and a liquid bottom product containing succinic acid and at least 20 wt % of water to prevent formation of amide by-products. A polar high-boiling solvent may be used in this step. Next, the bottom product is cooled to form solid portion containing succinic acid. The solid is recovered and hydrogenated in the presence of hydrogenation catalyst to produce THF and/or GBL and/or BDO. Example 4 in this patent started with 80 gram of 36% diammonium succinate solution and 80 gram of triglyme. During the evaporation, an additional amount of 3300 g of water had to be gradually fed to the distilling mixture in order to prevent formation of side products, such as succinamic acid and succinimide. A total of 3313 g of distillate was taken in the end. Then the solution was cooled down to precipitate 7.1 g of solid. The solid had to be recrystallized again by adding 7.1 g of hot water and cooled down to produce 3.9 g of succinic acid. The succinic acid yield toward the crystal was calculated to be only 17%, and still contains 0.099 wt % of succinamic acid. This process has an economic disadvantage that so much water needs to be added to the reaction (3300 g water for 80 g of feed in this case) and has to be distilled off. If this process is scaled up to an industrial level, it will require large amount of thermal energy for evaporation.

U.S. Pat. Nos. 5,958,744 and 6,265,190 disclose a method that requires concentration of disodium succinate broth to 30% w/w and adjusting pH to 1.5-1.8 by adding ammonium, H₂SO₄ and NH₄HSO₄ to yield succinic acid and ammonium sulfate. At this pH range, the solubility of succinic acid is low causing it to precipitate out. The precipitated succinic acid is redissolved in methanol. To produce a pure succinic product, the methanol is evaporated and the succinic acid is recrystallized out. Ammonium sulfate is insoluble in methanol, so methanol is added into ammonium sulfate crystallizer to help ammonium sulfate to crash out. The co-product ammonium sulfate can be cracked thermally, preferably at about 290-310° C. into ammonia and ammonium bisulfate. The evaporated methanol and water vapor could be condensed and reused. The quality of the succinic acid obtained according to the present invention in terms of impurities such as sulfate content is not known.

There is a need to produce succinic acid esters with minimal sulfur content in the range of 100 ppm, preferably less than 50 ppm and most preferably less than 10 ppm as the hydrogenation catalysts used in the conversion of succinate ester to its hydrogenated products such as BDO, GBL and THF are known to be sensitive to sulfur contamination.

The present invention provides a novel and simple route to convert a carbon source, such as glucose, via fermentation to succinic acid or one of its salts and then converting that product to dialkyl succinate. Dialkyl succinate can be hydrogenated leading to the production of BDO, GBL and THF. This novel method for recovering succinic acid from fermentation broth involves fractional crystallization step integrated with simulated moving bed chromatography. The production of bio-based dialkyl succinate ester, BDO, GBL, or THF via this method will have low carbon footprint and will help to expand the portfolio for value-added green chemicals.

### SUMMARY OF THE INVENTION

This present invention provides processes for preparing succinic acid and/or succinate ester from succinic acid salt in a fermentation broth.

Succinic acid salt suitable for the present invention is produced using bacterial and fungal biocatalysts including yeast. Biocatalysts for succinic acid production can utilize a variety of carbon sources including glucose, sucrose, glycerol and cellulosic hydrolysates. Succinic acid is accumulated in the fermentation broth as a salt having the counter ion selected from a group of alkaline metal, alkaline earth metal, ammonium, or alkylammonium group. Succinic acid is recovered from the fermentation broth comprising succinic acid salt as a free acid by means of acidifying the clarified fermentation broth using stronger acids. In one aspect of the present invention, a strong mineral acid such as sulfuric acid is used to treat the fermentation broth containing ammonium succinate leading to the release of succinic acid and ammonium sulfate. In another aspect of the present invention, a phosphoric acid is used to acidify the fermentation broth containing ammonium succinate leading to the release of succinic acid and ammonium phosphate.

Among various succinic acid salts that can be accumulated in a fermentation broth, ammonium succinate is preferentially used in the present invention. The processes according to the present invention enable maximum recovery of succinic acid from a fermentation broth in a cost-effective manner.

In one embodiment of the present invention, succinic acid and succinic acid ester are obtained using a process involving evaporation, filtration, acidification-associated double displacement reaction, fractional crystallization, separation of succinic acid crystals and mother liquor and recovering remaining succinic acid from mother liquor using simulated moving bed chromatography.

In one aspect of the present invention, the succinic acid recovered from simulated moving bed chromatography is further concentrated through evaporation and subjected to crystallization to obtain succinic acid crystals.

In another aspect of the present invention, the succinic acid crystals recovered through fractional distillation is separated by centrifugation and subject to esterification reaction to yield succinate ester which is recovered through fractional distillation while the succinic acid recovered from simulated moving bed chromatography is subjected to evaporation and crystallization procedure to recover succinic acid crystal.

In yet another aspect of the present invention, both the succinic acid crystal obtained from fractional crystallization step and the succinic acid crystals obtained through evaporation/crystallization process post-simulated moving bed chromatography are dissolved in methanol and subjected to esterification reaction. Succinate ester is recovered from methanolic solution through fractional distillation.

In one aspect of the present invention, the acidified fermentation broth is cooled to crystallize the succinic acid. In another embodiment of the present invention, the fermentation broth is subjected to evaporation before subjecting it to acidification. In a preferred aspect of this invention, the fermentation broth is subjected to evaporation and filtered before acidification step.

In another embodiment of the present invention, fermentation broth containing succinic acid salt is concentrated through evaporation followed by a salting out step. During the salting out process, the concentrated succinic acid salt is mixed with methanol to recover crystalline succinic acid salt as a precipitate and methanol-water phase retaining most of the impurities in the fermentation broth. Methanol is recovered from methanol-water phase by distillation for reuse. Succinic acid salt recovered as a solid precipitate is redissolved in water and subjected to a double displacement reaction with a strong acid to produce free succinic acid and newly formed ammonium salt of strong acid as the products of a double displacement reaction. The aqueous solution comprising succinic acid and newly formed ammonium salt is subjected to simulated moving bed chromatography to recover succinic and newly formed ammonium salt in different streams.

In one aspect of the present invention, sulfuric acid is used in the double displacement reaction to yield succinic acid and ammonium sulfate. In another aspect of the present invention, phosphoric acid is used in the double displacement reaction to yield succinic acid and ammonium phosphate.

In yet another embodiment of the present invention, succinic acid recovered from simulated moving bed chromatography is dissolved in methanol and subjected to esterification reaction followed by recovery of succinate ester through fractional distillation.

The succinic acid ester obtained according to the present invention is suitable for catalytic vapor-phase hydrogenation procedure to yield hydrogenation products such as BDO, GBL, and THF.

### BRIEF DESCRIPTION OF THE DRAWINGS

FIG. 1. Process flow diagram for the production of biomass-derived BDO, GBL and THF. In the fermentation process in this flow diagram, succinic acid is produced as ammonium succinate. Acidification is achieved by the addition of sulfuric acid leading to the release of free succinic acid which is recovered according to the process of the instant invention. In one aspect of the present invention, succinic acid resulting from acidification reaction is esterified and the succinate ester is recovered through fractional distillation. In another aspect of the present invention, succinic acid and ammonium sulfate resulting from acidification reaction are recovered in separate streams and the succinic acid thus recovered is subjected to polishing step to recover high purity succinic acid through evaporation and crystallization.

FIG. 2. Three flow sheet configurations for producing crude crystals of succinic acid. In the Flow sheet 1 configuration, fermentation broth containing ammonium succinate is acidified with the addition of sulfuric acid followed by concentration by evaporation leading to the recovery of acetic acid as a condensate. The acidified, concentrated fermentation broth is cooled leading to the crystallization of succinic acid. In the Flow sheet 2a configuration, fermentation broth containing ammonium succinate is concentrated by evaporation and ammonia is recovered in the condensate followed by cooling. Subsequently, the cooled concentrated fermentation broth is acidified and succinic acid is precipitated. In the Flow sheet 2b configuration, fermentation broth containing ammonium succinate is concentrated by evaporation and ammonia is recovered in the condensate. Sulfuric acid is used to acidify the concentrated fermentation broth and the succinic acid that precipitated out is heated and subjected to recrystallization to form larger and purer crystalline material. The acidification/precipitation, heating, and crystallization steps can all take place in single equipment, such as by using a draft-tube baffled crystallizer connected to a recirculation loop where sulfuric acid can be added. Heat liberated from acidification, and optionally an auxiliary heater, can be used to redissolve fine particles. The residence time and the mixing in the draft-tube baffled crystallizer are to be controlled so that large crystals with high purity are obtained. In all three configurations, the succinic acid is recovered at the end through filtration as crude crystals and the resulting mother liquor is subjected to further processing steps to recover remaining succinic acid.

FIG. 3. Solubility curve for ammonium sulfate in water. Ammonium sulfate is highly soluble in water. There is an increase in the solubility of ammonium sulfate in water with the increase in temperature. Even at 0° C., about 70 grams of ammonium sulfate is still soluble in 100 grams of water.

FIG. 4. Detailed process flow diagram for the separation of succinic acid and ammonium sulfate from acidified fermentation broth using simulated moving bed chromatography. Acidified fermentation broth is passed through simulated moving bed chromatography and succinic acid and ammonium sulfate are recovered in two different streams. The succinic acid steam is subjected to an optional polishing step, such as by nanofiltration and/or adsorption, followed by evaporation and crystallization process steps to recover succinic acid crystals with high purity. Alternatively, the succinic acid stream is subjected to evaporation leading to concentrated succinic acid stream which can be subjected to esterification reaction leading to the production of succinic acid esters suitable for hydrogenation reaction in the production of BDO, THF and GBL. The ammonium sulfate stream coming out of simulated moving bed chromatography is subjected to evaporation and crystallization process to produce ammonium sulfate crystals which can be dried to produce ammonium sulfate crystals suitable for use as fertilizer.

FIG. 5. Detailed process flow diagram for the separation of succinic acid and ammonium sulfate using fractional crystallization process integrated with simulated moving bed chromatography. According to this integrated process of the present invention, acidified fermentation broth is subjected to controlled crystallization process to yield succinic acid crystal and mother liquor with enriched ammonium sulfate content and remaining succinic acid. Succinic acid crystals from this fractional crystallization step can be dissolved in methanol and subjected to esterification reaction to yield dimethyl succinate which can be used as a substrate for the production of BDO, THF and GBL through hydrogenation. Mother liquor is subjected to simulated moving bed chromatography and ammonium sulfate and succinic acid are recovered in two different streams. Ammonium sulfate stream is subjected to evaporation, crystallization and drying steps to produce ammonium sulfate crystals suitable for use as fertilizer. The succinic acid stream is passed through a polishing step, which may include nanofiltration and/or ion-exchange and/or adsorption, evaporation, crystallization and drying steps to produce succinic acid with high purity.

### DETAILED DESCRIPTION OF THE PREFERRED EMBODIMENTS

As used in this invention, the term “bio-succinic acid” means succinic acid derived from renewable carbon sources through fermentation process involving biocatalysts. Succinic acid is accumulated in the fermentation broth as a succinic acid salt which is subjected to downstream processing to recover succinic acid.

As used in this invention, the term “bio-BDO” means BDO derived from hydrogenation reaction involving bio-succinic acid as a starting material. In the first step of the process for producing bio-BDO, bio-succinic acid is esterified to produce bio-succinic acid ester which in turn is used as a substrate in the hydrogenation reaction to yield bio-BDO.

As used in this invention, the term “evaporation” means subjecting an aqueous solution to elevated temperatures to reduce the water content of the aqueous solution. The evaporation process is preferentially carried out under vacuum,

As used in this invention, the term “concentration” means reducing the solvent content of a solution with reference to its solute content.

As used in this invention, the term “acidification” means adding an acid to an aqueous solution to reduce the pH of the said aqueous solution.

As used in this invention, the term “crystallization” means to form solid crystals precipitating from dissolved solute in an aqueous solution by means of varying the temperature or concentration of solute in the aqueous solution.

As used in this invention, the term “controlled crystallization” means to form solid crystals precipitating from dissolved solute in an aqueous solution by means of varying the temperature or concentration of the solute in the aqueous solution in a pre-defined rate.

As used in this invention, the term “filtration” means removal of particulate matter in a solution by means of passing the solution through a filter to retain the particulate matter. The filtration process is carried out under differential pressure across the membrane.

As used in this invention, the term “first crystallization” means the recovery of succinic acid from fermentation broth containing ammonium succinate following an acidification step and a concentration step. The acidification step involves the addition of an acid to the fermentation broth containing ammonium succinate to bring the pH of the fermentation broth to 2-2.5.

As used in this invention, the term “second crystallization” means the recovery of ammonium sulfate or ammonium phosphate from the mother liquor resulting from the removal of succinic acid in the first crystallization step. To begin with a fermentation broth containing ammonium succinate is used. When sulfuric acid is used in the acidification step, ammonium sulfate is recovered in the second crystallization step. On the other hand, when phosphoric acid is used in the acidification step, ammonium phosphate is recovered in the second crystallization step.

As used in this invention, the term “salting out” means precipitating ammonium succinate from a fermentation broth comprising dissolved ammonium succinate with the addition of methanol. The resulting ammonium succinate precipitate is recovered either through filtration or centrifugation.

As used in this invention, the term “salt splitting” means a double displacement reaction in which ammonium succinate is mixed with sulfuric acid in the presence of methanol. Presence of sulfuric acid causes the protonation of succinic acid from ammonium succinate accompanied by the formation of ammonium sulfate. Succinic acid resulting from double displacement reaction simultaneously dissolves and enters into an esterification reaction with methanol in the presence of sulfuric as a catalyst leading to the accumulation of dimethyl succinate. Depending on the rate of double displacement reaction and esterification reaction, varying amounts of succinic acid, ammonium sulfate mono-, and dimethyl succinate accumulate in the reaction mixture at the end of the salt splitting reaction.

As used in this invention, the term “double displacement reaction” means a chemical reaction in which the anions and cations exchange partners. For example, in a double displacement reaction involving ammonium succinate and sulfuric acid, a proton from sulfuric acid replaces the ammonium cation in ammonium succinate leading to the formation of succinic acid. Simultaneously, ammonium cation reacts with sulfate anion of sulfuric acid to form ammonium sulfate.

As used in this invention, the term “quality of succinic acid crystals” means relative purity of the succinic acid crystals recovered using the process according to the present invention. For example, the objective of the present invention is to recover succinic acid crystals from a fermentation broth containing ammonium succinate with minimal sulphur content so that there is no interference in the functioning of the catalysts that are useful in the further downstream processing leading to the production of BDO, THF and GBL.

The present invention provides the method for producing succinic acid and succinate ester from succinic acid salt present in a fermentation process. Succinate ester produced according to the present invention is useful in the production of BDO, GBL and THF

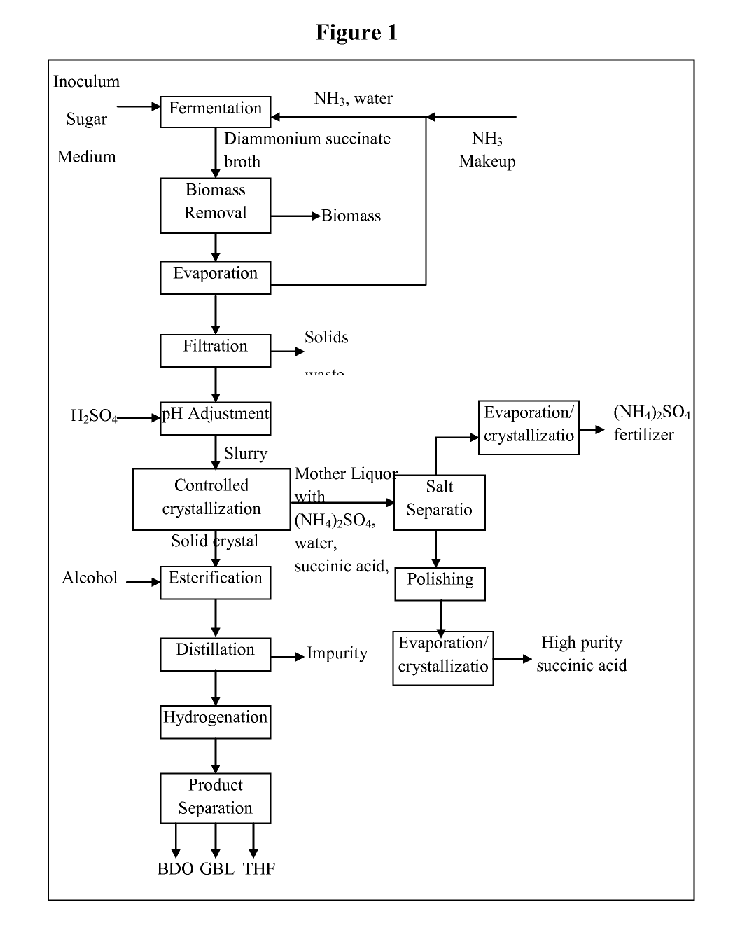

FIG. 1 provides a process flow diagram for the production of biomass-derived succinic acid, BDO, GBL, and THF. In a succinic acid producing fermentation suitable for the present invention, a variety of biocatalysts and a wide range of substrates suitable for microbial fermentation are used. The biocatalyst suitable for the present invention can be derived from a variety of microorganisms ranging from gram negative bacteria to fungi including yeast strains. The carbohydrate materials suitable for the present invention are glucose, sucrose, glycerol and lignocellulosic hydrolysates obtained from a variety of materials rich in six-carbon and five-carbon sugars. It is preferable to have biocatalysts with the ability to use both six-carbon and five-carbon sugars simultaneously. One desirable characteristic of any biocatalyst suitable for the present invention is that biocatalysts show a high titer and high yield for succinic acid production. The terms “succinic acid yield” and “yield” refer to the mole of the succinic acid produced per mole of carbohydrate material consumed. The terms “succinic acid titer” and “titer” as used in the present invention refer to the amount of succinic acid produced per unit volume of the fermentation broth per unit period of time (g/L/hr). Another desirable feature of the biocatalysts suitable for the present invention is the ability to grow in a minimal mineral salt medium without the need for any additional nutrient sources such as yeast extract or corn steep liquor. The fermentation process can be carried out either under anaerobic condition or aerobic condition or microaerobic condition.

In the biological fermentation process to produce succinic acid, inorganic alkali and trace nutrient chemicals are added to the fermentor to maintain the condition where the organisms can function optimally. For example, E. coli strain KJ122 obtained through genetic manipulations produces succinic acid at the highest yield when the pH is around 6.5-7.0. With the production of succinic acid during the fermentation process, the pH of the fermentation medium decreases significantly. In order to maintain the pH of the fermentation medium, bases such as potassium hydroxide, sodium hydroxide, and ammonium hydroxide are added gradually during the course of the fermentation. As a result, at the end of the fermentation process, the succinic acid accumulates in the fermentation medium as succinic acid salt. For example when ammonium hydroxide is used as the neutralizing base in the fermentation process involving KJ122 strain of E. coli, succinic acid accumulates at the end of fermentation in the form of diammonium succinate. According to the present invention, one could follow several different approaches to recover succinic acid from fermentation broth containing succinic acid salt and its subsequent chemical conversion into desired chemical entities such as BDO, GBL, and THF through appropriate chemical reactions.

The fermentation broth containing succinic acid in the form of a succinic acid salt is subjected to centrifugation and appropriate filtration steps to get rid of most of the particulate material in the fermentation broth including the cell mass and proteins. The centrifugation and filtration steps are referred as clarification step and the fermentation broth after clarification step is referred as clarified fermentation broth. In the recovery of succinic acid from fermentation broth containing a salt of succinic acid, an acidification step is followed. For example, to convert the dilute solution of ammonium succinate to succinic acid, a proton source needs to be provided. This can be achieved either by using an ion-exchange resin or by an acidification step.

When an ion-exchange resin is used in the recovery of succinic acid from fermentation broth containing salt of succinic acid, the succinate salt is split on the surface of the resin and depending on the charge on the resin either the succinate or its counter cation is retained on the surface of the resin. For example when cation ion-exchange resin is used and the resin is charged with proton, the proton from the resin is swapped with the succinate counter cation and succinic acid comes out in the effluent.

When the clarified fermentation broth containing a salt of succinic acid is acidified with a strong acid, free succinic acid and the corresponding salt accumulate. For example, when the fermentation broth containing ammonium succinate is acidified with sulfuric acid, free succinic acid and ammonium sulfate accumulate in the fermentation broth. Succinic acid needs to be separated from ammonium sulfate and the remaining solution, which primarily contains water and other impurities such as unconverted sugars, amino acids, and inorganic nutrients. There are several technologies that can be used to further purify the succinic acid.

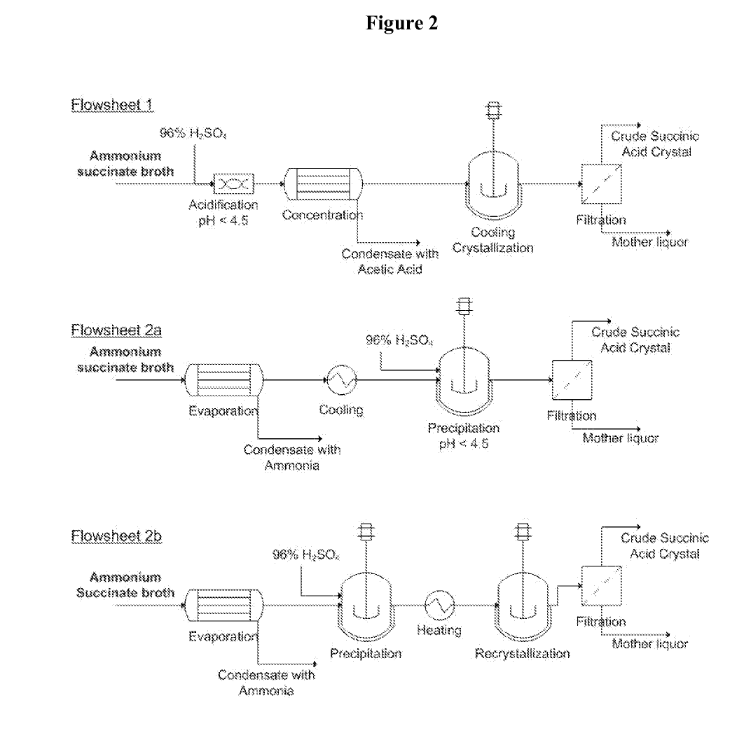

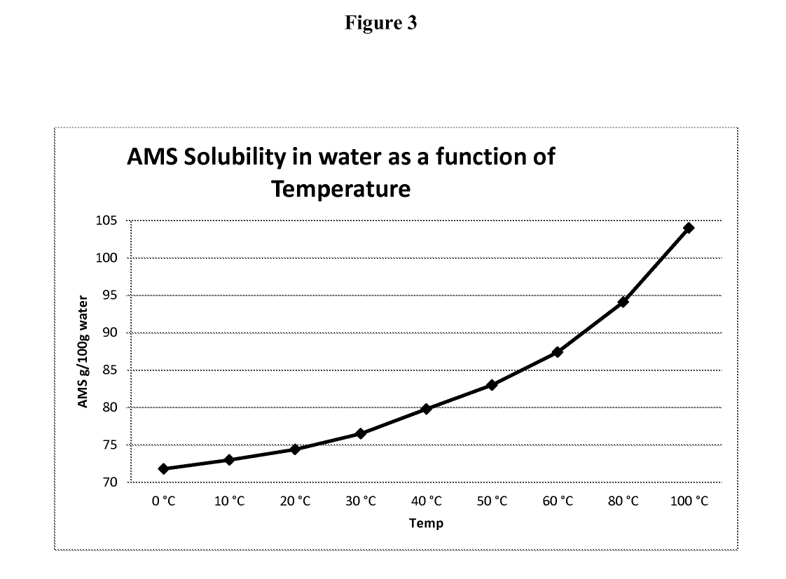

When a clarified fermentation broth containing ammonium succinate is acidified with a strong acid such as sulfuric acid or phosphoric acid, succinic acid and ammonium sulfate or ammonium phosphate accumulates in the aqueous phase. The aqueous phase is concentrated preferably under vacuum to remove water and volatile carboxylic acids such as acetic acid and formic acid. The solution is concentrated to preferably above 20 wt % of succinic acid and the crystallization of succinic acid is carried out by reducing the temperature of the mixture until its solubility limit at the corresponding temperature is lower than succinic acid concentration. While ammonium sulfate is highly soluble in aqueous solution (FIG. 3), the solubility of succinic acid in water is highly dependent on temperature. Succinic acid solubility decreases with the decrease in temperature. By means of controlling the cooling rate of the aqueous solution containing succinic acid, its solubility can be controlled leading to controlled crystallization of succinic acid. This crystallization step resulting in the formation of primarily crystalline succinic acid from the acidified fermentation broth is referred as “first crystallization” step (FIG. 2). The final slurry is separated from mother liquor using centrifugation and the succinic acid crystals are dried to remove moisture. If necessary, the succinic acid crystals can be dissolved and recrystallized to improve the purity of the succinic acid.

When phosphoric acid is used as an acidifying agent, ammonium phosphate is recovered as a co-product which can be used as a fertilizer or subjected to thermal degradation to yield phosphoric acid used in the process for recovering succinic acid from fermentation broth containing ammonium succinate.

The succinic acid thus recovered from fermentation broth through acidification process as described above is subjected to further downstream chemical processing as described below to obtain desired chemicals such as BDO, GBL and THF. The process begins when the succinic acid is subjected to esterification with alcohol in the presence of an esterification catalyst. In one aspect of the present invention, the esterification is carried out in the absence of any exogenous esterification catalyst and such an esterification process is referred as autocatalytic esterification reaction.

Dimethyl carbonate (DMC) has become very attractive for alkylation agent in the recent years as nontoxic and more environmentally benign alternative. This can be used for esterification of carboxylic acids instead of methanol at similar reaction performance levels. The byproduct of succinic acid esterification with DMC is CO₂, which can be recycled to succinic acid fermentation. DMC is proven to be highly chemo-selective to the desired ester product in case of hydroxy-carboxylic acids, such as salicylic acids, not affecting the hydroxyl group.

The esterification catalyst suitable for the present purpose may be a heterogeneous or homogeneous catalyst. The esterification reaction can be run using a variety of catalysts that include zeolites (X, Y, L, beta, ZSM-5, etc.), crystalline and amorphous metal oxides (silica and alumina), alkali modified zeolites (Na, K, Li, Cs, Ru, La, etc.), anion modified metal oxides and zeolites (SO₄, PO₄, BO₃, etc.), cation resins (Amberlyst-15, Amberlyst-70 etc.), alkaline hydroxides (NaOH, NH₄OH, KOH, etc.), alkaline alkoxides. The succinic acid ester thus produced can be purified further through fractional distillation. In one embodiment, butyl ester of succinic acid is produced first. Other types of esters such as methyl, ethyl and propyl esters of succinic acid can be obtained from butyl ester of succinic acid through transesterification process. When the esterification is done at the industrial scale, it may require the use of certain heterogeneous esterification catalysts. Under those conditions it is necessary to make sure that succinic acid feedstock to be used in the esterification reaction in the presence of such heterogeneous catalysts does not have contaminants that could interfere with the functioning of the esterification catalysts. For example, certain resins used in the industrial scale esterification of carboxylic acids are known to be sensitive to certain cation and anion contaminants of the carboxylic acid substrate and therefore it is necessary to make sure that the crystalline succinic acid used as the substrate in the esterification reaction is free of any contaminants that could interfere in the esterification reactions of the present invention.

According to the present process, succinic acid is esterified with an alcohol under super atmospheric pressure and at an elevated temperature substantially between 100° C. and 300° C. When a lower monohydric alcohol is used, the pressure should be high enough to minimize its vaporization, which would otherwise occur at such temperature, so that the reactions are kept in the liquid state in the reaction zone. A number of alcohols may be used in this esterification process, especially the methyl, ethyl, isopropyl, butyl, hexyl, octyl, and similar monohydric aliphatic compounds. It is preferable to run the reaction autocatalytically or use a homogeneous catalyst in the esterification reaction. Heterogeneous acid catalysts can be subjected to fouling when in contact with succinic acid stream that has not been purified to remove cations. Sulfuric acid, phosphoric acid, hydrochloric acid, and organic acids such as alkanesulfonic acid and arylsulfonic acids are suitable homogeneous catalysts. Water formed in the esterification reaction is removed by allowing the hot reaction mixture to pass from the reaction zone at elevated pressure into a zone at lower pressure, whereby water is flashed off.

In another embodiment of the present invention, simulated moving bed chromatography is integrated with the fractional crystallization process for recovering succinic acid and ammonium sulfate from an acidified fermentation broth containing succinic acid and ammonium sulfate.

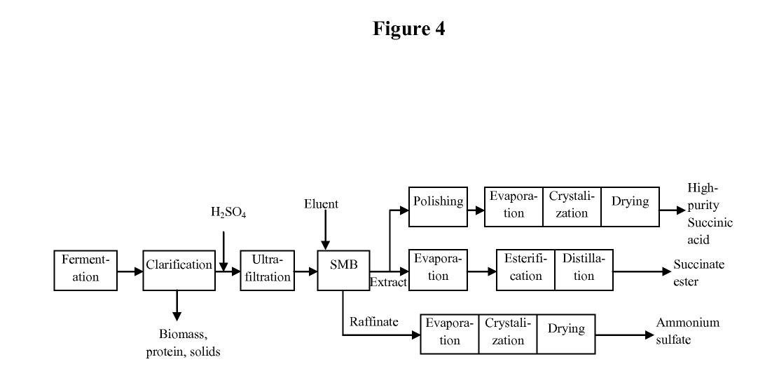

The traditional process for using simulated moving bed chromatography for recovering succinic acid from a fermentation broth containing ammonium succinate is illustrated in FIG. 4. The fermentation broth containing ammonium succinate is acidified to produce succinic acid and ammonium sulfate through double displacement reaction. The acidified fermentation broth is subjected to simulated moving bed chromatography and succinic acid and ammonium sulfate are recovered in two different streams. The ammonium sulfate stream is subjected to evaporation, crystallization and drying steps to yield ammonium sulfate crystals useful as fertilizer. The succinic acid stream is subjected to simple evaporation step to produce concentrated succinic acid solution which can be mixed with methanol and subjected to esterification reaction to yield dimethyl succinate which in turn can be used as a substrate to produce BDO, THF and GBL through hydrogenation reaction. Alternatively, the succinic acid stream is subjected to a polishing step, which may include nanofiltration and/or ion-exchange and/or adsorption, evaporation and crystallization steps to produce succinic acid crystals with high purity. The use of simulated moving bed chromatography in the recovery of succinic acid from fermentation broth containing ammonium succinate has been described in detail in the United States Patent Application Publication No. 2013/0096343 which has been incorporated herein in its entirety by reference.

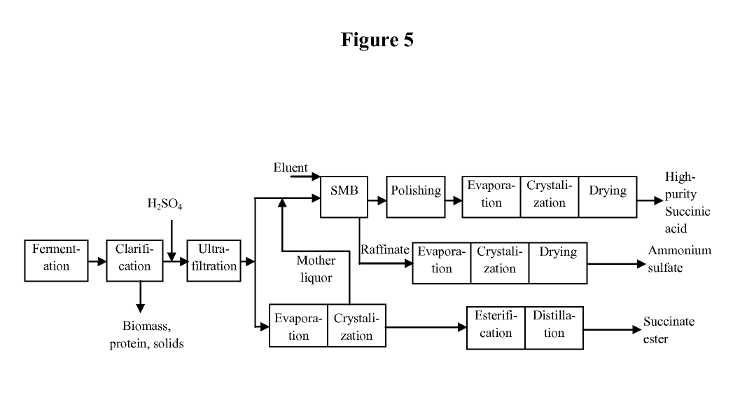

In the process according to the present invention, simulated moving bed chromatography is integrated with a fractional crystallization process to recover majority of succinic acid from the acidified fermentation broth as crude crystals before subjecting the mother liquor to simulated moving bed chromatography (FIG. 5). This integrated process according to the present invention provides cost saving opportunities in the recovery of succinic acid as it allows the recovery of significant amounts of succinic acid crystals through concentration and crystallization before subjecting much smaller mother liquor stream to simulated moving bed chromatography. According to one aspect of this integrated process, fermentation broth is acidified and concentrated through evaporation at elevated temperature under vacuum before fractional crystallization to recover succinic acid crystals from acidified fermentation broth. In a preferred aspect of the present invention, fermentation broth is first concentrated through evaporation at elevated temperature under vacuum before acidification to produce succinic acid and ammonium sulfate through double displacement reaction. There is an advantage in following this preferred evaporation—acidification process as the amount of acid required in the acidification reaction is reduced and the succinic acid crystals resulting from fractional crystallization step is also found to be of higher purity.

In the integrated process for the recovery of succinic acid according to the present invention, succinic acid resulting from the controlled crystallization process is separated from mother liquor through centrifugation. The resulting mother liquor is processed through simulated moving bed chromatography. Succinic acid and ammonium sulfate in the mother liquor are recovered in two different liquid streams from simulated moving bed chromatography. Succinic stream from simulated moving bed chromatography is processed through an optional polishing step, which may include nanofiltration and/or ion-exchange and/or adsorption, evaporation, crystallization and drying steps to recover succinic acid crystals with high purity. The ammonium sulfate stream from simulated moving bed chromatography is subjected to evaporation, crystallization and drying to produce ammonium sulfate crystals suitable for use as fertilizer.

The embodiments described above have been provided only for the purpose of illustrating the present invention and should not be treated as limiting the scope of the present invention. The chemical reaction schemes depicted herein are just examples. There may be many variations to these chemical reaction schemes or the steps or operations described therein without departing from the spirit of the invention. Numerous modifications of the embodiments described herein may be readily suggested to one of skilled in the art without departing from the scope of the appended claims. It is intended, therefore, that the appended claims encompass such modifications to the embodiments disclosed herein.

The following examples are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

### EXPERIMENTAL SECTION

### General Remarks

Strain and Inoculum Preparations:

KJ122 (E. coli C, ΔldhA, ΔadhE, ΔackA, ΔfocA-pflB, ΔmgsA, ΔpoxB, ΔtdcDE, ΔcitF, ΔaspC, ΔsfcA) was used in the present invention. KJ122 was derived from E. coli C (ATCC 8739) strain through genetic modifications as described by Jantama et at (2008a; 2008b) and in the International Patent Applications published under Patent Cooperation Treaty with International Publication Nos. WO 2008/115958 and WO 2010/115067. All these documents are herein incorporated by reference.

E. coli strain KJ122 is capable of fermenting 10% glucose in AM1 mineral media to produce 88 g/L succinate, normalized for base addition, in 72 hours. AM1 medium contains 2.63 g/L (NH₄)₂HPO₄, 0.87 g/L NH₄H₂PO₄, 1.5 mM MgSO₄, 1.0 mM betaine, and 1.5 ml/L trace elements. The trace elements are prepared as a 1000× stock and contained the following components: 1.6 g/L FeCl₃, 0.2 g/L CoCl₂.6H₂O, 0.1 g/L CuCl₂, 0.2 g/L ZnCl₂.4H₂O, 0.2 g/L NaMoO₄, 0.05 g/L H₃BO₃, and 0.33 g/L MnCl₂.4H₂O. The pH of the fermentation broth is maintained at 7.0 with: 1:4 (6 N KOH:3 M K₂CO₃) (1.2 N KOH, 2.4 M K₂CO₃).

In some experiments, corn steep liquor was added. It is a byproduct from the corn wet-milling industry. When compared to the yeast extract and peptone, it is an inexpensive source of vitamins and trace elements.

Organic Acid and Sugar Analysis:

The concentration of various organic acids and sugars were measured by HPLC. Succinic acid, sugars, and other organic acids present in the fermentation broth were analyzed on an Agilent 1200 HPLC apparatus with a BioRad Aminex HPX-87H column. A BioRad Microguard Cation H⁺ was used as a guard column. The standards for HPLC analysis were prepared in 0.008N sulfuric acid. The HPLC column temperature was maintained at 50° C. Sulfuric acid at 0.008N concentration was used as a mobile phase at the flow rate of 0.6 ml/min. Quantification of various components was done by measuring their absorption at 210 nm. Quantification of sugars and other components was done using a refractive index detector.

Determination of Sulfur, and Phosphorous Content Using Inductively Coupled Plasma Optical Emission Spectrometry (ICP-OES).

Samples derived from various processes according to the present invention can be tested using Inductively Coupled Plasma Optical Emission Spectrometry. The samples are diluted to less than 5% organics using 2% trace metal grade nitric acid. The ICP-OES produces a curve that ranges from 0.05 ppm to 10 ppm, thus the sample is diluted so that the target molecule concentration falls between these concentrations. If the sample contains any complex matrices or insoluble liquids, they are digested or ashed prior to being dissolved. The samples are then placed in an autosampler connected to the ICP-OES, with a quality control in the first, middle, and last position. The ICP-OES will then analyzed the standards, generate a calibration curve, and analyze the samples. The software will then calculate the ppm for each substance detected, and from there the initial concentration of the substance can be determined based upon the dilution factor when the sample was prepared.

Determination of Chlorides, Sulfates, Phosphates and Ammonium Using Ion Chromatography (IC).

The concentration of ammonium and various anions can be quantified using Ion Chromatography. Samples were analyzed using a Dionex 1100 Ion Chromatography instrument with an AS-DV autosampler. Standards from 0.04 to 30 ppm are prepared in deionized water and a calibration curve is generated prior to each run. Samples are diluted so the analyte of interest falls within the calibration curve. For ammonium analysis a Dionex CS16 column is used with a CG16 guard column and a CSRS400 suppressor. 35 mM Methanesulfonic acid is used as an eluent. For chloride, sulfate and phosphate analysis a Dionex AS17-HC column is used with a AG17-HC guard column and a ASRS400 suppressor. 28 mM Sodium Hydroxide is used as an eluent for cation analysis. Quantification is done by measuring the conductivity response of each component.

Determination of Amino Acids.

Amino acids are measured by HPLC. Samples were analyzed on an Agilent 1100 HPLC with online derivatization and a fluorescence detector. A Phenomenex Gemini C18 column is used with a phosphate buffer and solvent gradient to elute the compounds. Samples and standards are diluted with 0.1M hydrochloric acid and derivatized with OPA reagent prior to injection. Quantification of amino acids is done by measuring the response from a fluorescence detector with excitation at 338 nm and emission at 450 nm.

### Example 1

### Fermentative Production of Succinic Acid

E. coli strain KJ122 was inoculated in a minimum media consisting of NBS, 100 mM MOPS, 2% glucose, 1 mM MgSO₄, trace element and 0.1 mM CaCl₂ at 37° C. Once the cell density reached OD=5, the inoculums were transferred to a fermenter containing initial medium consisting of 25 mM KH₂PO₄, 3 mM MgSO₄, 2 mM betaine, and 8 ppm of antifoam 204. Fermentation was run in a fed-batch mode with glucose as the carbohydrate source. As succinic acid was being produced, a solution of 7M NH₄OH and 3M NH₄HCO₃ was metered into the fermentor to maintain the pH at around 6.5-7 and to provide a source for CO₂. After 48 hours, the fermentation was completed. Biomass was removed by a tangential flow microfiltration unit. The filtered broth composition is shown in Table 1.

### Example 2

### Acidification of Fermentation Broth and Recovery of Succinic Acid Crystals—1

Diammonium succinate fermentation broth prepared in Example 1 was used in this experiment. 49 ml of 36.25N sulfuric acid was used to acidify 1200 ml of fermentation broth to pH 2.0. Then 1010 g of the acidified broth was evaporated in a rotary evaporator under vacuum at 70° C. to obtain 330 g of concentrated broth. The condensate was analyzed and found to contain 0.8 g/L of acetic acid. The concentrated broth was cooled down by a step-wise cooling profile at the rate of −5° C. every 30 minutes in an orbital shaker set at 200 rpm. The crystal was filtered under vacuum to obtain 55.75 g of wet crystals. The crystal was subsequently washed with 25 ml of deionized water and dried overnight at 50° C. This process corresponds to the process illustrated in the FIG. 2 as “Flow sheet 1”. The recovery of succinic acid as the crystal was calculated to be 53%. The composition of dry crystal was analyzed and is shown in Table 2. The crystals showed very high succinic acid purity of 95.9%. The impurities consist of 0.17 wt % ammonium ion and 0.22 wt % sulfur.

### Example 3

### Acidification of Fermentation Broth and Recovery of Succinic Acid Crystals—2

Diammonium succinate fermentation broth prepared in Example 1 was used in this experiment. 1038 g of the broth was concentrated in a rotary evaporator to 416 g under vacuum. Some brown solids precipitated out of the solution. The condensate was analyzed and was found to contain 2.8 g/L of ammonia. The concentrated broth was divided into three portions. Portion 1 (99.92 g) was filtered to remove solids and then acidified with 8 ml of 36.25N H₂SO₄ to pH 2.0. Portion 2 (84.66 g) was acidified with 8 ml of 36.25N H₂SO₄ to pH 2.0. Portion 3 was saved as a reference. During the acidification step, white precipitate started to form at pH 5.2 and more came out of solution as the pH dropped further. After letting the solution cool to ambient room temperature, the precipitates from portions 1 and 2 were filtered and each was washed with 10 ml of deionized water. The solids were dried overnight at 50° C. Compositional analysis of these solids is shown in Table 2. Compared with Example 2, this process configuration uses less sulfuric acid to bring the solution pH to 2.0. This process corresponds to the process illustrated in the FIG. 2 as “Flow sheet 2a”. It is believed that since ammonia was removed as a condensate, less acid is required to reduce the solution pH, which is very desirable from an economic standpoint. However, the purity of the crystal was lowered. Crystal Portion 1, which had been filtered after evaporation showed 90 wt % succinic acid purity, while crystal Portion 2 showed only 82 wt % purity. The filtration step after evaporation clearly helped purify the crystals substantially; the amount of ammonium ion, sulfur, phosphorus, potassium ion, and amino acid are substantially less.

### Example 4

### Acidification of Fermentation Broth and Recovery of Succinic Acid Crystals—3

Diammonium succinate fermentation broth prepared in Example 1 was used in this experiment. 1010 g of the broth was concentrated under reduced pressure at 70° C. in a rotary evaporator to 264 g. The condensate was analyzed and found to contain 2.1 g/L of ammonia. Some brown solids precipitated out of solution. However, the broth was not filtered. The solution was acidified with 34.85 ml of 36.25N H₂SO₄ to the pH of 2.07. Then, a controlled crystallization was performed in an orbital shaker by cooling at the rate of −5° C. every 30 minutes to room temperature. The crystal was filtered and washed with 40 ml of deionized water and then dried at 50° C. overnight. The crystal composition is shown in Table 2. By doing a proper controlled cooling crystallization step, the succinic acid crystal had a surprisingly very high purity of 99 wt % succinic acid even without a filtration step after evaporation. The amount of reduction in ammonium ion, sulfur, phosphorus, and amino acid is greater than 10 fold. This process corresponds to the process illustrated in the FIG. 2 as “Flow sheet 2b”.

The sulfuric acid usages and the crystal qualities from three flow sheets are compared in Table 2. Results showed that Flow sheet 2a and 2b used less sulfuric acid than that of Flow sheet 1 to adjust the pH to 2. In terms of the crystal purity, Flow sheet 2a had the highest amount of impurity incorporated into the final product. This is likely due to the fact that succinic acid precipitated out in an uncontrolled manner and the mother liquor carrying impurities was trapped inside the precipitate. With a recrystallization step in Flow sheet 2b, the crystal quality was much improved. The results suggest that it is desirable to concentrate the sample first followed by acidification and recrystallization.

### Example 5

### Effect of Temperature on Succinamic Acid Formation

A 10 wt % aqueous solution of synthetic diammonium succinate was prepared by dissolving reagent grade succinic acid in water and then aqueous ammonia was added to the solution. 45 ml of the solution was charged under atmospheric pressure into a 75 ml vessel of Multi-Parr reactor model 5000. The solution was heated up to various temperatures and held for a period of time. After that the solution was immediately cooled down by running cooling water through a cold finger. Then the reactor content was emptied and the composition of the product was analyzed. The results are shown in Table 3. These results showed that increasing temperature and the exposure duration can increase the formation of amide by-products. The process according to this invention recommends the evaporation process to be conducted under reduced pressure, which should be advantageous in reducing the yield loss of succinic acid to amides.

### Example 6

### Crude Crystallization of Succinic Acid in Large Volume Fermentation Broth

Liquid samples 1 and 2 were obtained from a large volume fermentation (85,000 liters) run using KJ122 strain as described in the Example 1. Liquid sample 1 is diammonium succinate fermentation broth after centrifugation to separate cell mass. Liquid sample 2 is diammonium succinate fermentation broth after centrifugation and an acidification step with sulfuric acid to pH 4.5. Approximately 45 kg of each liquid sample was used to generate crude succinic crystal using the following procedures: (1) approximately 45 kg of each sample was concentrated ˜3× via evaporation at reduced pressure. (2) Concentrated ammonium succinate was filtered by a filter press using 5-micron and 0.5-micron membranes. (3) The filtered material was then partitioned into 4 kg batches for acidification to pH≈2 with 96% H₂SO₄; this was conducted in 5 L Pyrex bottles using a stir bar for agitation. (4) Heating and recrystallization was executed in an Innova-43 incubator shaker at 150 rpm and an initial temperature of 70° C. The incubator shaker was programmed to drop the temperature by 10° C. per hour in order to carry out slow cooling. (5) Precipitated crystals and mother liquor from each acidification/crystallization batch were pooled together in a 20 L Jacketed Filter Reactor to accomplish filtration. Vacuum was used to pull mother liquor through a Teflon filter into a container. Crystals were washed with deionized water and then left to dry for several hours under vacuum. (6) After drying, the crystals were harvested from the reactor. Mass balance is summarized in Table 4. The compositions of the crude crystals are shown in Tables 5 and 6.

TABLE 1
Composition of Fermentation Broth

| | |
|---|---|
| Succinic Acid (g/L) | 78.2 |
| Acetic Acid (g/L) | 0.6 |
| Lactic Acid (g/L) | — |
| Pyruvic Acid (g/L) | 0.04 |
| Malic Acid (g/L) | — |
| Fumaric Acid (g/L) | 0.06 |
| Formic Acid (g/L) | — |
| Ammonium ion (g/L) | 23.0 |
| Chloride ion (ppm) | 2.5 |
| Total sulfur (ppm) | 110 |
| Total phosphorus (ppm) | 318 |
| Amino acids (g/L) | 22.6 |

TABLE 2
Product quality of succinic acid crystals

| | Flow sheet 1 | Flow sheet 2a | | Flow sheet 2b |
|---|---|---|---|---|
| Process Configuration | Acidify → | Evaporate → | Evaporate → | Evaporate → |
| | Evaporate | Filter → | Acidify & | Acidify & |
| | → | Acidify & | Precipitate | Precipitate → |
| | Crystallize | Precipitate | | Recrystallize |
| H₂SO₄ usage g/g broth | 0.0746 | 0.0639 | | 0.0635 |
| Moisture (wt %) | 0.24% | 0.21% | 0.22% | 0.42% |
| Succinic acid (wt %) | 95.9% | 90.0% | 82.3% | 99.2% |
| Fumaric acid (wt %) | 0.07% | 0.06% | 0.05% | 0.11% |
| Succinamic acid (wt %) | 0.18% | 0.67% | 0.73% | 0.42% |
| Succinimide (wt %) | not detected | not detected | not detected | not detected |
| Succindiamide (wt %) | not detected | not detected | not detected | not detected |
| Amino acids (wt %) | 0.39% | 3.86% | 4.51% | 0.14% |
| Ammonium (ppmw) | 1769.8 | 11411 | 17751 | 130.6 |
| Sulfur (ppmw) | 2248.8 | 19308 | 23631 | 1082.8 |
| phosphorus (ppmw) | 76.0 | 825.9 | 915.3 | not detected |
| Chloride (ppmw) | not detected | 8.45 | 7.45 | not detected |
| Calcium (ppmw) | 22.6 | 3.6 | 4.1 | not detected |
| Potassium (ppmw) | 612.0 | 272.3 | 432.7 | 29.94 |
| Magnesium (ppmw) | 12.5 | 4.5 | 7.0 | not detected |
| Sodium (ppmw) | 7.8 | 22.9 | 33.6 | not detected |

TABLE 3
Effect of temperature on succinamic acid formation

| | Run Number | | | | |
|---|---|---|---|---|---|
| | MA13 | MA15 | MA16 | MA17 | MA18 |
| Temperature (° C.) | 150 | 120 | 120 | 90 | 90 |
| Duration (hrs) | 4 | 4 | 8 | 4 | 8 |
| Yield | | | | | |
| Succinimide | 0.13% | 0.04% | 0.13% | 0.00% | 0.00% |
| Succindiamide | 0.08% | 0.00% | 0.00% | 0.00% | 0.00% |
| Succinamic acid | 10.7% | 0.7% | 1.6% | 0.00% | 0.07% |

TABLE 4
Mass balance for succinic acid samples 1 and 2 obtained from
crude crystallization of large volume fermentation broth

| | | | Total | Total | SAC in | |
|---|---|---|---|---|---|---|
| | Liquid | SAC | SAC in | Crude | crude | |
| | feed | g/L in | feed | crystal | crystal | % SAC |
| | (kg) | feed | (kg) | (kg) | (kg) | recovered |
| Sample 1 | 42.5 | 68.38 | 2.84 | 2.32 | 2.18 | 76.8% |
| Sample 2 | 45.2 | 68.09 | 3.00 | 2.72 | 2.59 | 86.3% |

TABLE 5
Composition of crude succinic crystals

| | | Crude 1 | Crude 2 |
|---|---|---|---|
| Color (YI) | | 19.51 | 15.83 |
| Color (WI) | | −7.39 | 1.46 |
| Moisture | wt % | 6.47% | 0.71% |
| Organics | | | |
| Succinic acid | wt % | 95.91% | 96.98% |
| Acetic Acid | wt % | 0.092% | 0.075% |
| Pyruvic Acid | wt % | 0.006% | 0.028% |
| Malic Acid | wt % | not detected | not detected |
| Fumaric Acid | wt % | 0.004% | 0.009% |
| Lactic Acid | wt % | not detected | not detected |
| Glucose | wt % | 0.016% | 0.019% |
| Maltose | wt % | 0.050% | 0.030% |
| Glycerol | wt % | not detected | not detected |
| Formic Acid | wt % | not detected | not detected |
| Succinamic Acid | wt % | 0.03% | 0.05% |
| Succinimide | wt % | not detected | not detected |
| Succindiamide | wt % | not detected | 0.02% |

TABLE 6
Composition of crude succinic crystals (Anions and
Cations)

| | | Crude 1 | Crude 2 |
|---|---|---|---|
| Anions | | | |
| Sulphate | ppm | 11267.51 | 9053.58 |
| Phosphate | ppm | not | not detected |
| | | detected | |
| Chlorides | ppm | 3.39 | 0.51 |
| Cations | | | |
| Ammonium | ppm | 5747.57 | 4650.70 |
| As | ppm | not | not detected |
| | | detected | |
| Ca | ppm | 1.73 | 0.91 |
| Cu | ppm | 0.52 | not detected |
| Fe | ppm | not | 1.12 |
| | | detected | |
| K | ppm | 143.30 | 100.75 |
| Mg | ppm | 9.18 | 6.22 |
| Mn | ppm | not | not detected |
| | | detected | |
| Ni | ppm | not | not detected |
| | | detected | |
| Pb | ppm | not | not detected |
| | | detected | |
| Zn | ppm | 0.39 | not detected |
| Na | ppm | 2.13 | 3.68 |
| Cr | ppm | not | not detected |
| | | detected | |
| S | ppm | 4264.90 | 3471.00 |
| P | ppm | 102.72 | 74.14 |
| Total Amino Acid | ppm | 253.1 | 181.18 |

### REFERENCES

All references are listed for the convenience of the reader. Each reference is incorporated by reference in its entirety.
U.S. Pat. No. 2,565,487
U.S. Pat. No. 2,722,541
U.S. Pat. No. 2,766,273
U.S. Pat. No. 4,100,189
U.S. Pat. No. 4,405,717
U.S. Pat. No. 4,444,881
U.S. Pat. No. 4,645,855
U.S. Pat. No. 4,885,247
U.S. Pat. No. 5,034,105
U.S. Pat. No. 5,068,418
U.S. Pat. No. 5,132,456
U.S. Pat. No. 5,143,834
U.S. Pat. No. 5,168,055
U.S. Pat. No. 5,210,296
U.S. Pat. No. 5,323,639
U.S. Pat. No. 5,352,825
U.S. Pat. No. 5,405,992
U.S. Pat. No. 5,449,824
U.S. Pat. No. 5,453,365
U.S. Pat. No. 5,502,240
U.S. Pat. No. 5,510,526
U.S. Pat. No. 5,641,406
U.S. Pat. No. 5,766,439
U.S. Pat. No. 5,773,653
U.S. Pat. No. 5,780,276
U.S. Pat. No. 5,780,678
U.S. Pat. No. 5,786,185
U.S. Pat. No. 5,892,109
U.S. Pat. No. 5,958,744
U.S. Pat. No. 5,986,133
U.S. Pat. No. 6,043,392
U.S. Pat. No. 6,087,532
U.S. Pat. No. 6,160,173
U.S. Pat. No. 6,187,951
U.S. Pat. No. 6,229,046
U.S. Pat. No. 6,265,190
U.S. Pat. No. 6,280,985
U.S. Pat. No. 6,291,708
U.S. Pat. No. 6,319,382
U.S. Pat. No. 6,320,077
U.S. Pat. No. 6,342,626
U.S. Pat. No. 6,384,276
U.S. Pat. No. 6,403,825
U.S. Pat. No. 6,452,0151
U.S. Pat. No. 6,472,559
U.S. Pat. No. 6,476,239
U.S. Pat. No. 6,478,965
U.S. Pat. No. 6,509,179
U.S. Pat. No. 6,518,454
U.S. Pat. No. 6,534,079
U.S. Pat. No. 6,583,310
U.S. Pat. No. 6,660,505
U.S. Pat. No. 6,641,734
U.S. Pat. No. 6,664,413
U.S. Pat. No. 6,670,505
U.S. Pat. No. 6,772,213
U.S. Pat. No. 6,803,217
U.S. Pat. No. 6,872,314
U.S. Pat. No. 6,926,810
U.S. Pat. No. 6,942,803
U.S. Pat. No. 7,019,170
U.S. Pat. No. 7,026,145
U.S. Pat. No. 7,144,977
U.S. Pat. No. 7,186,856
U.S. Pat. No. 7,238,837
U.S. Pat. No. 7,297,809
U.S. Pat. No. 7,439,392
U.S. Pat. No. 7,498,450
U.S. Pat. No. 7,528,277
U.S. Pat. No. 7,601,865
U.S. Pat. No. 7,632,479
U.S. Pat. No. 7,652,167
U.S. Pat. No. 7,696,375
U.S. Pat. No. 7,709,689
U.S. Pat. No. 7,754,916
U.S. Pat. No. 7,820,859
U.S. Pat. No. 7,915,447
U.S. Pat. No. 8,084,626
U.S. Pat. No. 8,193,375
U.S. Pat. No. 8,203,021
U.S. Pat. No. 8,241,482
U.S. Pat. No. 8,293,935
U.S. Pat. No. 8,338,145
U.S. Pat. No. 8,410,306
U.S. Patent Application Publication No. 2004/0210087
U.S. Patent Application Publication No. 2006/0252956
U.S. Patent Application Publication No. 2006/0276674
U.S. Patent Application Publication No. 2006/0041165
U.S. Patent Application Publication No. 2007/0014895
U.S. Patent Application Publication No. 2007/0096481
U.S. Patent Application Publication No. 2007/0161816
U.S. Patent Application Publication No. 2007/0167649
U.S. Patent Application Publication No. 2008/0287714
U.S. Patent Application Publication No. 2008/042958
U.S. Patent Application Publication No. 2009/0076297
U.S. Patent Application Publication No. 2009/0104322
U.S. Patent Application Publication No. 2009/0234160
U.S. Patent Application Publication No. 2010/0297715
U.S. Patent Application Publication No. 2001/0137843
U.S. Patent Application Publication No. 2011/0257429
U.S. Patent Application Publication No. 2011/0300589
U.S. Patent Application Publication No. 2012/0142945
U.S. Patent Application Publication No. 2012/0238722
U.S. Patent Application Publication No. 2013/0018206
U.S. Patent Application Publication No. 2013/0096343
U.S. Patent Application Publication No. 2013/0081947
U.S. Patent Application Publication No. 2013/0096343
International Patent Application Publication No. WO2005/051885
International Patent Application Publication No. WO2009/030395
International Patent Application Publication No. WO2009/040095
International Patent Application Publication No. WO2009/082050
International Patent Application Publication No. WO2010/022960
International Patent Application Publication No. WO2010/022966
International Patent Application Publication No. WO2011/064151
International Patent Application Publication No. WO2011/069007
International Patent Application Publication No. WO2011/123269
International Patent Application Publication No. WO2011/160760
International Patent Application Publication No. WO2013/012590
Benedict, D. J., Parulekar, S. J. and Tsai, S-P. (2006) Pervaporation-assisted esterification and succinic acids with downstream ester recovery. J. Mem. Sci. 281: 435-445.
Budarin, V., Luque, R., Macquarrie, D. J. and Clark, J. H. (2007) Towards a bio-based industry: Benign catalytic esterification of succinic acid in the presence of the water. Chem. Euro. J. 13: 6914-6919.
Cheng, Ke-ke., Zhao, X-B., Zeng, J., Wu, R-C., Xu, Y-Z., Liu, D-H., Zhang, J-A. (2012) Downstream processing of biotechnological produced succinic acid. 95: 841-850.
Davis, S. 1, 4-Butanediol, CEH Market Research Report, October 2010.
Davison, B. H., Nghiem, N. P. and Richardson, G. L. (2004) Succinic acid adsorption from fermentation broth and regeneration. App. Biochem. Biotechnol. 113-116: 653-669.
Delhomme, C., Weuster-Botz, D., Kuhn, F. E. (2009) Succinic acid from renewable resources ad a C4 building-block chemical—A review of the catalytic possibilities in aqueous media. Green Chem. 11: 13-26.
Delhomme, C. (2011) Process integration of fermentation and catalysts for the production of succinic acid derivatives. Dissertation submitted to degree of Doctor of Philosophy in Engineering, Technische Universitat Munchen, Germany.
Delhomme, C., Goh, S. L. M., Kuhn, F. E. and Weuster-Botz, D. (2012) Esterification of bio-based succinic acid in biphasic systems: Comparison of chemical and biological catalysts. J. Mol. Cata. Enz. 80: 39-47.
Filachione, E. M., Costello, E. J. and C. H. Fisher. (1951) Preparation of esters by reaction of ammonium salts with alcohols. J. Amer. Chem. Society. 13(11), 5265-5267.
Filachione, E. M. and Costello, E. J. (1952) Lactic esters by reaction of ammonium lactate with alcohols. Ind. & Eng. Chem., 44, 2189-2191.
Huh, Y. S., Jun, Y-S., Hong, Y. K., Song, H., Lee, S. Y., Hong, W. H. (2006) Effective purification of succinic acid from fermentation broth produced by Mannheimia succiniciproducens. Proc. Biochem. 41: 1461-1465.
Jafar, J. J., Budd, P. M., Hughes, R. (2002) Enhancement of esterification reaction yield using zeolite Avapour permeation membrane. J. Mem. Sci. 199: 117-123.
Jantama, K., Haupt, M. J., Svoronos, S. A., Zhang, X., Moore, J. C., Shanmugham, K. T., Ingram, L. O. (2008) Combining metabolic engineering and metabolic evolution to develop nonrecombinant strains of E. coli C that produce succinate and malate. Biotech Bioeng 99: 1140-1153.
Jantama, K., Zhang, X., Moore, J. C., Shanmugham, K. T., Svoronos, S. A., Ingram, L. O. (2008) Eliminating side products and increasing succinate yields in engineered strains of Escherichia coli C. Biotech. Bioeng. 101: 881-893.
Jun, Y-S., Huh, Y. S., Hong, W. H. and Hong, Y. K. (2005) Kinetics of the extraction of succinic acid with tri-octylamine in 1-octanol solution. Biotechnol. Prog. 21: 1673-1679.
Jun, Y-S., Lee, E. Z., Huh, Y. S., Hong, Y. K. (2007) Kinetic study for the extraction of succinic acid with ZTOA in fermentation broth; effects of pH, salt and contaminated acid. Biochem. Eng. J. 36: 8-13.
Kurzrock, T. and Weuster-Botz, D. (2009) Recovery of succinic acid from fermentation broth. Biotechnol. Lett. DOI 10.1007/s10529-009-0163-6. Published online: 8 Nov. 2009.
Kurzrock, T. and Weuster-Botz, D. (2011) New reactive extraction systems for separation of bio-succinic acid. Bioprocess Biosyst. Eng. 34: 779-787.
Li, Q., Wang, D., Wu, Y., Li, W., Zhang, Y., Xing, J. and Su, Z. (2010) One Step recovery of succinic acid from fermentation broths by crystallization. Sep. Purif. Technol. 72: 294-300.
Li, Q., Li, W-l., Wang, D., Lilu, B-b., Tang, H., Yang, M-h., Liu, Q-f., Xing, J. M. and Su, Z-g. (2010) pH Neutralization while succinic acid adsorption onto anion-exchange resins. Appl Biochem Biotechnol. 160:438-445.
Lin, C. S. K., Luque, R., Clark, J. H., Webb, C. and Du, C. (2011) Wheat-based biorefining strategy for fermentative production and chemical transformations of succinic acid. Biofuels, Bioprod. Bioref. 6: 88-104.
Londono, A. O. (2010) Separation of succinic acid from fermentation broths and esterification by a reactive distillation method. A dissertation submitted for the degree of Doctor of Philosophy in Chemical Engineering, Michigan State University, USA.
Lopez-Garzon, C. S., Ottens, M., van der Wielen, A. M. and Straathof, A. J. J. (2012) Direct downstream catalysis: From succinate to its diethyl ester without intermediate acidification. Chem. Eng. J. 200-202: 637-644.
Luque, R., Lin, C. S. K., Du, C., Macquarrie, D. J., Koutinas, A., Wang, R., Webb, C. and Clark, J. H. (2009) Chemical transformations of succinic acid recovered from fermentation broths by a novel direct vacuum distillation-crystallization method. Green Chem. 11: 193-200.
Minh, D. P., Besson, M., Pinel, C., Fuertes, P. and Petitjean, C. (2010) Aqueous-phase hydrogenation of biomass-based succinic acid to 1,4-butanediol over supported bimetallic catalysts. Top. Catal. 53: 1270-1273.
Orjuela, A., Kolah, A., Lilra, C. T. and Miller, D. J. (2011) Mixed succinic acid/acetic acid esterification with ethanol by reactive distillation. Ind. Eng. Chem. Res. 50: 9209-9220.
Orjuela, A., Yanez, A., Peereboom, L., Lira, C. T. and Miller, D. J. (2011) A novel process for recovery of fermentation-derived succinic acid. Sep. Purif. Tech. 83: 31-37.
Orjuela, A., Yanez, A. J., Santhanakrishnan, A., Lira, C. T. and Miller, D. J. (2012) Kinetics of mixed succinic acid/acetic acid esterification with Amberlyst 70 ion exchange resin as catalyst. Chem. Eng. J. 188: 98-107.
Orjuela, A., Kolah, A., Hong, X., Lira, C. T. and Miller, D. J. (2012) Diehtyl succinate synthesis by reactive distillation. Sep. Pur Tech. 88: 151-162.
Orjuela, A., Orjuela, A., Lira, C. T. and Miller, D. J. (2013) A novel process for recovery of fermentation-derived succinic acid: Process design and economic analysis. Bioreso. Tech. 139: 235-241.
Rahman, M. B. A., Jarmi, N. I., Chaibakhsh, N. and Basri, M. (2011) Modeling and optimization of lipase-catalysed production of succinic acid ester using central composite design analysis. J. Ind. Microbiol. Biotechnol. 38: 229-234.
Song, H., Huh, Y. S., Lee, S. Y., Hong, W. H. and Hong, Y. K. (2007) Recovery of succinic acid produced by fermentation of a metabolically engineered Mannheimia succiniciproducens strain. J. Biotechnol. 132: 445-452.
Varadarajan, S. and Miller, D. J. (1999) Catalytic upgrading of fermentation-derived organic acids. Biotechnol. Prog. 15: 845-854
Wang, C., Li, Q., Tang, H., Zhou, W., Yan, D., xing, J. and Wan, Y. (2012) Clarification of succinic acid fermentation broth by ultrafiltration in succinic acid bio-refinery. J. Chem. Technol. Biotechnol. 88: 444-448.
Zhang, X., Jantama, K., Moore, J. C., Shanmugam, K. T., Ingram, L. O. (2009) Metabolic evolution of energy-conserving pathways for succinate production in Escherichia coli. Proc. Natl. Acad. Sci. U.S. A. 106: 20180-20185.

What is claimed:

1. A process for preparing succinic acid comprising the steps of:
a. fermentation of carbohydrate substrates using a biocatalyst to produce a succinate salt;
b. clarification of fermentation broth to remove biocatalyst, proteins, and insolubles;
c. concentration of said clarified fermentation broth from step (b);
d. acidification of concentrated fermentation broth from step (c);
e. controlled crystallization of succinic acid in the acidified and concentrated fermentation broth from step (d);
f. separating crystalline succinic acid from mother liquor; and
g. subjecting said mother liquor to simulated moving bed chromatography to recover remaining succinic acid; and
h. crystallizing said succinic acid recovered in step (g).

2. A process for preparing succinic acid as in claim 1 wherein said acidification of concentrated fermentation broth is carried out using sulfuric acid.

3. A process for preparing succinate ester comprising the steps of:
a. fermentation of carbohydrate substrates using a biocatalyst to produce a succinate salt;
b. clarification of fermentation broth to remove biocatalyst, proteins, and insolubles;
c. concentration of said clarified fermentation broth from step (b);
d. acidification of concentrated fermentation broth from step (c);
e. controlled crystallization of succinic acid in the acidified and concentrated fermentation broth from step (d);
f. separating crystalline succinic acid from mother liquor;
g. dissolving said crystalline succinic acid from step (f) in methanol;
h. esterification of said succinic acid dissolved in methanol in step (g);
i. subjecting said mother liquor from step (f) to simulated moving bed chromatography to recover remaining succinic acid;
j. crystallizing said succinic acid recovered in step (i);
k. dissolving crystalline succinic acid from step (j) in methanol;
l. esterifying said succinic acid dissolved in methanol in step (k); and
m. recovering succinate ester from steps (h) and (l) through fractional distillation.

4. A process for preparing succinic acid as in claim 3 wherein said acidification of concentrated fermentation broth is carried out using sulfuric acid.

5. A process for preparing succinate ester as in claim 3 wherein sulfur content of said succinate ester is below 50 ppm level.

6. A process for preparing succinate ester as in claim 3 wherein sulfur content of said succinate ester is below 10 ppm level.

7. A process for preparing succinic acid and succinate ester comprising the steps of:
a. fermentation of carbohydrate substrates using a biocatalyst to produce a succinate salt;
b. clarification of fermentation broth to remove biocatalyst, proteins, and insolubles;
c. concentration of said clarified fermentation broth from step (b);
d. acidification of concentrated fermentation broth from step (c);
e. controlled crystallization of succinic acid in the acidified and concentrated fermentation broth from step (d);
f. separating crystalline succinic acid from mother liquor;
g. dissolving said crystalline succinic acid crystal form step (f) in methanol;
h. esterification of said succinic acid dissolved in methanol in step (g);
i. recovering succinate ester from step (h) through fractional distillation;
j. subjecting said mother liquor from step (f) to simulated moving bed chromatography to recover remaining succinic acid; and
k. crystallizing said succinic acid from step (j).

8. A process for preparing succinic acid as in claim 7 wherein said acidification of concentrated fermentation broth is carried out using sulfuric acid.
